(19) United States
(12) Patent Application Publication
(10) Pub. No.: US 2012/0180796 A1
(43) Pub. Date: Jul. 19, 2012

(54) Tracheostomy Tubes

(75) Inventors: Timothy Bateman, Kent (GB); Daniel Jay Coates, Ogden Dunes, IN (US)

(73) Assignee: Smiths Group plc, London (GB)

(21) Appl. No.: 13/430,857

(22) Filed: Mar. 27, 2012

Related U.S. Application Data
(63) Continuation-in-part of application No. 11/886,586, filed on Sep. 18, 2007.

(30) Foreign Application Priority Data
Mar. 19, 2005 (GB) 0505724.5

(51) Int. Cl. A61M 16/04

(52) U.S. Cl. 128/207.14

(57) ABSTRACT

A fenestrated tracheostomy tube has two rows of openings extending along the outside of the curve of the tube where they will be located in the trachea. The shaft of the tube is made of a soft material, such as silicone, and is reinforced along the fenestrated region by a frame of a stiffer material. The frame has a longitudinal spine extending between the two rows and has several ribs extending from the spine circumferentially on opposite sides of the openings.

This invention relates to tracheostomy tubes.

Tracheostomy tubes are used to provide a passage for ventilation gas to the trachea of a patient through a surgically-made opening in the throat. The tubes may be of various different kinds. The tube may simply comprise an extruded or moulded hollow plastics shaft shaped appropriately for the patient's anatomy. Alternatively, the tube may be reinforced by means of a helical metal wire or a stiff, helical plastic element. A tube reinforced in this way is protected against radial crushing forces and helps prevent kinking. This enables a softer, more flexible plastics to be used, which enables the tube itself to be relatively flexible.

Where the tube is made of a soft material, such as silicone, it can be particularly difficult to provide fenestration openings in the tube because this weakens the wall of the tube in the fenestrated region.

It is an object of the present invention to provide an alternative tracheostomy tube.

According to one aspect of the present invention there is provided a tracheostomy tube having a shaft with a patient end, a machine end and a fenestrated region along its length including a plurality of openings spaced along the fenestrated region and adapted to lie within the trachea, the shaft being formed of a relatively soft material, the fenestrated region being strengthened by a structural member of a stiffer material having a longitudinal member and a plurality of rib members extending circumferentially of the structural member spaced along the longitudinal member, and at least two of the rib members extending on opposite sides of two said openings.

Preferably the structural member extends along only the fenestrated region. Preferably the shaft is of a silicone material and the structural member is of a plastics material. Preferably at least one of the rib members extends between adjacent ones of the openings, one of the rib members extends above an opening at an upper end of the fenestrated region and one of the rib members extends below an opening at a lower end of the fenestrated region.

According to another aspect of the present invention there is provided a tracheostomy tube having a shaft with a patient end, a machine end and a fenestrated region along its length including a plurality of openings arranged in two rows spaced along the fenestrated region and adapted to lie within the trachea, the shaft being formed of a relatively soft material, the fenestrated region being strengthened by a structural member of a stiffer material having a longitudinal member and a plurality of rib members extending circumferentially of the structural member spaced along the longitudinal member, the longitudinal member extending between the two rows of openings and at least one of the rib members extends between adjacent ones of the openings.

A tracheostomy tube according to the present invention, will now be described, by way of example, with reference to the accompanying drawing, in which:

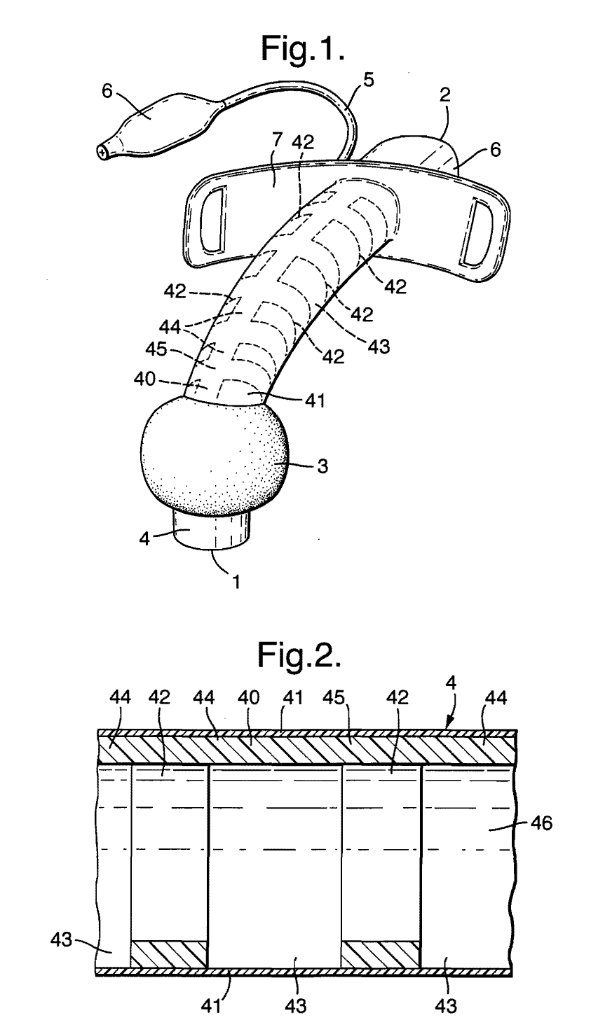

FIG. 1 is a perspective view of a first form of tube;

FIG. 2 is an enlarged cross-sectional side elevation view along a part of the length of the tube of FIG. 1;

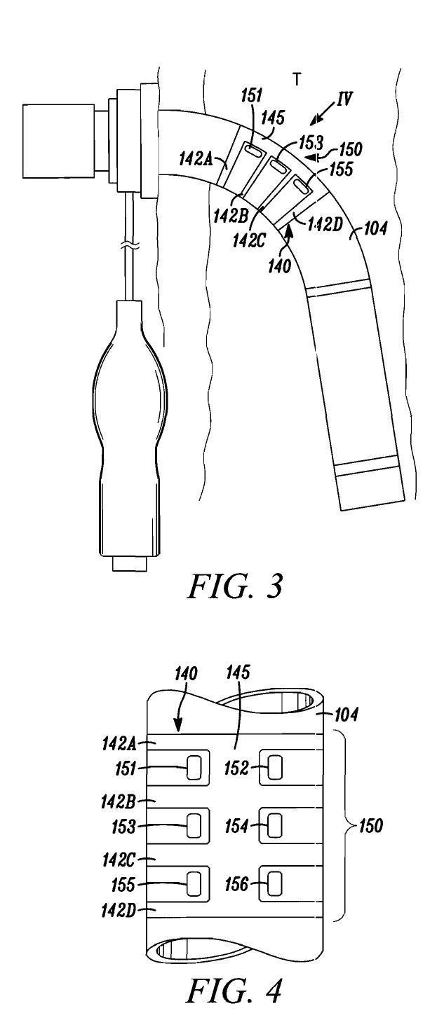

FIG. 3 is a perspective view of an alternative form of tube located in the trachea with the cuff in a deflated state; and

FIG. 4 is an enlarged view of a part of the tube of FIG. 3 along the arrow IV in FIG. 3.

With reference first to FIGS. 1 and 2, the tube is curved along its length, to conform to the anatomy of the patient, from its forward, patient end 1 to its rear, machine end 2. Alternatively, the tube could be straight and sufficiently flexible to conform to the shape of the anatomy. Towards its patient end 1 the tube has a large-volume, low-pressure sealing cuff 3 embracing the outside of a shaft 4. The cuff 3 is inflatable and deflatable, in the usual way, by means of an inflation line 5 having a balloon and connector 6 at its machine end. At its rear end 2, the tube has a standard 15 mm connector 6 and a neck flange 7.

As so far described, the tube is conventional. Where the tube differs from previous tubes is that the tube shaft 4 is made of two separate components, namely a skeletal, structural member or frame 40 and a sheath 41 attached to the outside of, and covering, the frame. The outside of the sheath 41, in use, contacts tissue of the tracheostomy.

The frame 40 is an integral, one-piece moulding of a relatively stiff plastics material, such as polycarbonate. The frame 40 comprises nine annular ribs or rings 42, only six of which are shown in the drawing, which extend circumferentially around the tube as complete circles. Alternatively, the ribs need not be complete circles and could be present in different numbers. The ribs 42 are spaced equally from one another along the length of the shaft 4 and are separated from one another by gaps 43. The ribs 42 are supported relative to one another by longitudinal members 44, which in the present example are arranged in a line to form a single spine 45 extending in axial alignment along the outside of the curve of the shaft 4. The spine 45 could include a conduit extending along its length to provide an air passage (not shown) between the inflation line 5 and the interior of the cuff 3. In other examples, the longitudinal members need not be aligned with one another but could, for example, be staggered around the shaft. The continuous nature of the spine 45, in the present arrangement, gives the shaft 40 a relatively high strength to resist axial compression forces, which can be met during insertion through neck tissue. It also helps define the curve and plane of curvature of the shaft 4. The frame 40 has a wall thickness of around 0.75 mm, which is the same for the ribs and the longitudinal members, although the thickness of the ribs and longitudinal members could be different. The width of the ribs and longitudinal members is greater than their thickness, typically being about 5 mm.

The sheath 41 is in the form of a thin film of a flexible plastics material, such as plasticised PVC and has a wall thickness of 0.3 mm, making it substantially thinner than the frame 40. The sheath 41 may be assembled on the frame 40 by overmoulding or dipping. It will be appreciated that the materials used for the frame 40 and the sheath 41 should be compatible so that the sheath bonds securely to the frame. The thickness of the shaft 4 in the region of the film 41 is considerably less than in conventional tracheostomy tubes, which have a wall thickness around 2 mm. Even the frame 40 itself is thinner than the wall of conventional tubes, which is possible because the material from which it is made is relatively hard compared with conventional tubes. In some arrangements the sheath could have the same thickness as the frame and the sheath may be moulded about the frame so that the frame is incorporated into the thickness of the wall of the sheath.

The tube is inserted through a surgically made opening into the trachea in the usual way with the external surface of the sheath 41 contacting the neck tissue through the opening and with the internal surface of the sheath and the frame 40 providing a bore 46 for passage of ventilation gas into and out of the trachea. The frame 40 gives the tube sufficient axial rigidity and the desired degree of flexibility. The frame 40 enables a very low wall thickness compared with conventional tubes yet with the necessary mechanical properties. The thin wall enables the diameter of the bore 46 through the tube to be maximised for a minimum external diameter, thereby minimizing trauma to the patient. The small thickness of the sheath 41 may result in a slightly ribbed external surface feel to the shaft but this is not believed to make insertion significantly more difficult.

The frame arrangement can have particular advantages in a fenestrated tracheostomy tube, that is, a tracheostomy tube formed with one or more small openings in its side wall, so that a part of the patient's breathing passes through these openings and via his nose or mouth, instead of through the machine end of the tracheostomy tube. The fenestration openings can be closed by means of an inner cannula, which is removed when it is desired to allow gas to flow through the openings. The inner cannula could be replaced by an inner cannula with openings that align with the openings in the outer tube. Such fenestration openings also enable patients to talk since exhaled gas can be allowed to flow to the larynx via the openings. Examples of fenestrated tubes are described in GB1522632, U.S. Pat. No. 4,852,565, U.S. Pat. No. 5,771,888, U.S. Pat. No. 6,722,367, U.S. Pat. No. 5,957,978 and U.S. Pat. No. 7,987,851. Such fenestrated tubes can be provided satisfactorily but, where the tube is of a soft material, such as silicone, the removal or absence of the material to create the fenestrations can weaken the tube in the fenestrated region to the extent that the tube could be prone to buckling and collapse in this region. FIGS. 3 and 4 show an arrangement by which such a tube can be reinforced in the fenestrated region. Components equivalent to those in the tube shown in FIGS. 1 and 2 are given the same reference numbers with the addition of 100.

The frame or structural member 140 comprises a longitudinal member or spine 145 and four annular or circumferential ribs 142A, 142B, 142C and 142D spaced along the length of the spine. The frame 140 could be of a relatively stiff plastics material such as a thermoplastic or thermosetting plastics material although the frame need not be of a plastics material but could, for example, be of a non-ferrous metal. The frame 140 could extend along the entire length of the tube but is preferably confined to a fenestrated region 150 along the tube where reinforcement is needed so that the remainder of the tube retains its soft, flexible characteristic. The frame 140 is embedded within or otherwise attached to the shaft 104 of the tube, which is thicker than the sheath shown in the arrangement in FIGS. 1 and 2. The shaft 104 is moulded or otherwise formed from a relatively soft plastics material, such as silicone. The thickness and nature of the material of the shaft 104 is such as to give it sufficient strength along most of its length apart from the region 150 where shaft material has been removed or is absent from six fenestrations, holes or openings 151 to 156 arranged in two rows along the shaft. This fenestrated region 150 is located approximately midway along the tube where the holes 151 to 156 will lie midway in the trachea T facing upwards, that is, towards the patient's larynx and mouth. Typically the holes 151 to 156 are elongated along the axis of the shaft 104 with rounded ends and are about 4 mm long and 2 mm wide, although the size and number of the holes will vary according to the size of the tube. The total area of the holes 151 to 156 is preferably about 10% greater than the cross-sectional area of the interior of the shaft 104. The frame 140 extends along the fenestrated region 150 with its spine 145 extending longitudinally along the outside curve of the tube between the two rows of holes 151, 153 and 155, and 152, 154 and 156. The two central ribs 142B and 142C extend between adjacent holes and the end ribs 142A and 142D extend above and below the upper holes 151 and 152 and lower holes 155 and 156 at the upper and lower ends of the fenestrated region 150 respectively.

By incorporating the frame 140 into the shaft 104 the fenestrations 151 to 156 can be provided safely in tubes of a soft material.

What we claim is:

1. A tracheostomy tube having a shaft with a patient end, a machine end and a fenestrated region along its length including a plurality of openings spaced along the fenestrated region and adapted to lie within the trachea, wherein the shaft is formed of a relatively soft material, wherein the fenestrated region is strengthened by a structural member of a stiffer material having a longitudinal member and a plurality of rib members extending circumferentially of the structural member spaced along the longitudinal member, and wherein at least two of the rib members extend on opposite sides of two said openings.

2. A tracheostomy tube according to claim 1, wherein the structural member extends along only the fenestrated region.

3. A tracheostomy tube according to claim 1, wherein the shaft is of a silicone material.

4. A tracheostomy tube according to claim 1, wherein the structural member is of a plastics material.

5. A tracheostomy tube according to claim 1, wherein at least one of said rib members extends between adjacent ones of the openings.

6. A tracheostomy tube according to claim 1, wherein one of the rib members extends above an opening at an upper end of the fenestrated region.

7. A tracheostomy tube according to claim 1, wherein one of the rib members extends below an opening at a lower end of the fenestrated region.

8. A tracheostomy tube having a shaft with a patient end, a machine end and a fenestrated region along its length including a plurality of openings arranged in two rows spaced along the fenestrated region and adapted to lie within the trachea, wherein the shaft is formed of a relatively soft material, wherein the fenestrated region is strengthened by a structural member of a stiffer material having a longitudinal member and a plurality of rib members extending circumferentially of the structural member spaced along the longitudinal member, wherein the longitudinal member extends between the two rows of openings and at least one of the rib members extends between adjacent ones of the openings.

9. A tracheostomy tube according to claim 8, wherein the structural member extends along only the fenestrated region.

10. A tracheostomy tube according to claim 8, wherein the shaft is of a silicone material.

11. A tracheostomy tube according to claim 8, wherein the structural member is of a plastics material.

12. A tracheostomy tube according to claim 8, wherein one of the rib members extends above an opening at an upper end of the rows of openings

13. A tracheostomy tube according to claim 8, wherein one of the rib members extends below an opening at a lower end of the rows of openings.
